(19) United States
(12) Patent Application Publication
(10) Pub. No.: US 2019/0090961 A1
(43) Pub. Date: Mar. 28, 2019

(54) SYSTEMS AND METHODS FOR ABLATION VISUALIZATION

(71) Applicant: COVIDIEN LP, MANSFIELD, MA (US)

(72) Inventors: DARREN G. GIROTTO, LOUISVILLE, CO (US); KEVIN J. FRANK, LOUISVILLE, CO (US)

(21) Appl. No.: 16/142,580

(22) Filed: Sep. 26, 2018

Related U.S. Application Data
(60) Provisional application No. 62/563,694, filed on Sep. 27, 2017.

(51) Int. Cl. A61B 34/20; A61B 18/00; A61B 90/00

(52) U.S. Cl. CPC A61B 34/20; A61B 18/00; A61B 2034/107; A61B 2090/3762; A61B 2018/00577; A61B 90/37

(57) ABSTRACT

Systems and methods for ablation visualization are disclosed. In various embodiments, a system for ablation visualization includes a display device and a computing device communicatively coupled to the display device. The computing device receives an indication of a location within a patient, accesses CT image data associated with the patient, where the CT image data includes image data for the location within the patient, and receives an orientation of an ablation probe having a central axis. Based on the CT image data, the computing device generates at least two images of at least two mutually orthogonal views of the anatomy of the patient encompassing the location within the patient. The at least two images include a probe-axial view of the anatomy of the patient, where the probe-axial view is orthogonal to the central axis of the ablation probe. The computing device communicates the at least two images to a display device to be displayed on the display device.

### CROSS REFERENCE TO RELATED APPLICATIONS

The present application claims the benefit of, and priority to, U.S. Provisional Patent Application Ser. No. 62/563,694 filed on Sep. 27, 2017, the entire contents of which are incorporated herein by reference.

### BACKGROUND

### Technical Field

The present disclosure relates to visualizing a treatment procedure and, more particularly, to systems and methods for visualizing the anatomy of a patient based on the position and orientation of an ablation probe.

### Description of Related Art

Computed tomography (CT) images are commonly used to identify objects, such as physiological structures, in a patient's body. In particular, CT images can be used by physicians to identify malignant tissue or problematic structures in a patient's body and to determine their location within the body. Once the location is determined, a treatment plan can be created to address the problem, such as planning a pathway into the patient's body to remove of the malignant tissue or procedures for accessing and altering the problematic structures. Ablation of tumors is an example of a more targeted approach to tumor treatment. In comparison to traditional body-wide types of cancer treatment, such as chemotherapy, ablation technologies are much more targeted and limited but are just as effective. Thus, such approaches are beneficial in providing targeted treatment that limits unnecessary injury to non-problematic tissue or structures in the patient's body, but they require the assistance of more complex technical tools. Accordingly, there continues to be interest in developing further technical tools to assist with targeted treatment of tissue or structural problems in a patient's body.

### SUMMARY

Provided in accordance with embodiments of the present disclosure are systems and methods for visualizing the anatomy of a patient based on the position and orientation of an ablation probe.

In an aspect of the present disclosure, a system for ablation visualization includes a display device and a computing device communicatively coupled to the display device. The computing device includes a processor and a memory. The memory stores instructions which, when executed by the processor, cause the computing device to receive an indication of a location within a patient, access CT image data associated with the patient, where the CT image data includes image data for the location within the patient, and receive an orientation of an ablation probe having a central axis. Based on the CT image data, the computing device generates two or more images of two or more mutually orthogonal views of the anatomy of the patient encompassing the location within the patient. The two or more images include a probe-axial view of the anatomy of the patient, wherein the probe-axial view is orthogonal to the central axis of the ablation probe. In various embodiments, the two or more images further include patient-centric axial, sagittal, and/or coronal views encompassing the location within the patient. The computing device communicates the two or more images to the display device for display on the display device.

In various embodiments, the computing device, based on the CT image data, further generates an image of a probe-sagittal view of the anatomy of the patient encompassing the location within the patient and an image of a probe-coronal view of the anatomy of the patient encompassing the location within the patient. The probe-axial view, the probe-sagittal view, and the probe-coronal view are mutually orthogonal. In various embodiments, the computing device generates a composite image including the probe-axial view, the probe-sagittal view, and the probe-coronal view, and communicates the composite image to the display device for display on the display device.

In various embodiments, the computing devices generates the probe-axial view, the probe-sagittal view, and the probe-coronal views of the anatomy of the patient to include a depiction of the ablation probe with the orientation and positioned at the location within the patient.

In various embodiments, the computing device generates, based on the CT image data, a three-dimensional depiction of the anatomy of the patient encompassing the location within the patient. In various embodiments, the computing device generates the three-dimensional depiction to include a depiction of the ablation probe with the orientation and positioned at the location within the patient. In various embodiments, the computing device generates the three-dimensional depiction of the anatomy of the patient to be within a cubical space. In various embodiments, the computing device presents to a user a choice to select one or more of the probe-axial view, the probe-sagittal view, the probe-coronal view, the three-dimensional depiction, or the three-dimensional cubical space, for display on the display device. In various embodiments, the computer device further presents to a user a choice to select one or more of a patient-centric axial view, sagittal view, or coronal view, for display on the display device.

In various embodiments, the computing device receives a planning operation for an ablation procedure based on the CT image data, where the indication of the location within the patient and the orientation of the ablation probe are received during the planning operation and are based on the CT image data. In various embodiments, the planning operation includes an operation to add an ablation target and/or an operation to add an ablation zone.

In various embodiments, the indication of the location within the patient and the orientation of the ablation probe are based on a real-time location and a real-time orientation of an ablation probe within the patient during a medical procedure.

In an aspect of the present disclosure, a method for ablation visualization includes receiving an indication of a location within a patient, accessing CT image data associated with the patient, where the CT image data includes image data for the location within the patient, and receiving an orientation of an ablation probe having a central axis. Based on the CT image data, the method generates two or more images of two or more mutually orthogonal views of the anatomy of the patient encompassing the location within the patient. The two or more images include a probe-axial view of the anatomy of the patient, wherein the probe-axial view is orthogonal to the central axis of the ablation probe. In various embodiments, the two or more images further include patient-centric axial, sagittal, and/or coronal views encompassing the location within the patient. The method communicates the two or more images to a display device for display on the display device.

In various embodiments, the method, based on the CT image data, further generates an image of a probe-sagittal view of the anatomy of the patient encompassing the location within the patient, and an image of a probe-coronal view of the anatomy of the patient encompassing the location within the patient. The probe-axial view, the probe-sagittal view, and the probe-coronal view are mutually orthogonal. In various embodiments, the method includes generating a composite image including the probe-axial view, the probe-sagittal view, and the probe-coronal view, and communicates the composite image to the display device for display on the display device.

In various embodiments, the method can generate the probe-axial view, the probe-sagittal view, and the probe-coronal views of the anatomy of the patient to include a depiction of the ablation probe with the orientation and positioned at the location within the patient.

In various embodiments, the method includes generating, based on the CT image data, a three-dimensional depiction of the anatomy of the patient encompassing the location within the patient. In various embodiments, the method can generate the three-dimensional depiction to include a depiction of the ablation probe with the orientation and positioned at the location within the patient. In various embodiments, the method generates the three-dimensional depiction of the anatomy of the patient to be within a cubical space. In various embodiments, the method includes presenting to a user a choice to select one or more of the probe-axial view, the probe-sagittal view, the probe-coronal view, the three-dimensional depiction, or the three-dimensional cubical space, for display on the display device. In various embodiments, the computer device further presents to a user a choice to select one or more of a patient-centric axial view, sagittal view, or coronal view, for display on the display device.

In various embodiments, the method includes receiving a planning operation for an ablation procedure based on the CT image data, where the indication of the location within the patient and the orientation of the ablation probe are received during the planning operation and are based on the CT image data. In various embodiments, the planning operation includes an operation to add an ablation target and/or an operation to add an ablation zone.

In various embodiments, the indication of the location within the patient and the orientation of the ablation probe are based on a real-time location and a real-time orientation of an ablation probe within the patient during a medical procedure.

### BRIEF DESCRIPTION OF THE DRAWINGS

Various aspects and features of the present disclosure are described hereinbelow with references to the drawings, of which:

FIG. 1A is a diagram of an ablation probe and a probe-sagittal plane that is relative to the ablation probe;

FIG. 1B is a diagram of the ablation probe of FIG. 1 and a probe-coronal plane that is relative to the ablation probe;

FIG. 1C is a diagram of the ablation probe of FIG. 1 and a probe-axial plane that is relative to the ablation probe;

FIG. 2 is a block diagram of an exemplary system for ablation visualization in accordance with aspects of the present disclosure;

FIG. 3 is a diagram of exemplary regions of a patient to which the disclosed systems and methods may be applied;

FIG. 4 is an exemplary display interface showing an axial view in accordance with aspects of the present disclosure;

FIG. 5 is an exemplary display interface showing a coronal view in accordance with aspects of the present disclosure;

FIG. 6 is an exemplary display interface showing a sagittal view in accordance with aspects of the present disclosure;

FIG. 7 is an exemplary display interface showing a composite view in accordance with aspects of the present disclosure; and

FIG. 8 is a flow diagram of an exemplary operation of a system in accordance with aspects of the present disclosure.

### DETAILED DESCRIPTION

The present disclosure provides systems and methods for visualizing the anatomy of a patient based on the position and orientation of an ablation probe. In various embodiments, views of the anatomy are probe-centric such that the views shift and rotate as an ablation probe is moved or rotated. In various embodiments, the views of the anatomy further include patient-centric views such as views of the anatomy that are aligned with the patient's axial, coronal, and sagittal planes and which shift as the probe is moved, but which do not rotate as the probe is rotated. The systems and methods present a clinician with enhanced visualization during ablation treatment planning, including ablation target identification and selection, target sizing, treatment zone sizing, entry point and route selection, and treatment plan review. The system also presents a clinician with the capability to visualize the position of an ablation probe in real time during an ablation treatment procedure, and also compare pre-operative and post-operative CT image data to assess the outcome of an ablation treatment procedure.

As persons skilled in the art will recognize, most medical imaging systems are patient-centric and define axial, coronal, and sagittal views with respect to the orientation of the patient's body. This typical approach is generally used for viewing images of a patient to identify and determine the location and orientation of a region of interest. However, while patient-centric axial, coronal, and sagittal views are informative and allow a physician to become oriented in the surgical cavity of a patient, they can present blind spots that may be problematic when actually treating the patient. The present disclosure supplements body-centric medical imaging views by providing medical imaging views that are instrumentation centric. In the case of the instrument being an ablation probe, medical imaging views are generated based on the position and orientation of the ablation probe in place of or in addition to medical imaging views based on the orientation of the patient's body. As described below herein, the probe-centric views can include three mutually orthogonal views of the ablation probe and the tissue it is interacting with, which may not line up with the typical patient-centric axial, coronal, and sagittal views. The probe-centric views described herein are more information for determining whether and how an ablation probe will impact or is impacting tissue of the patient.

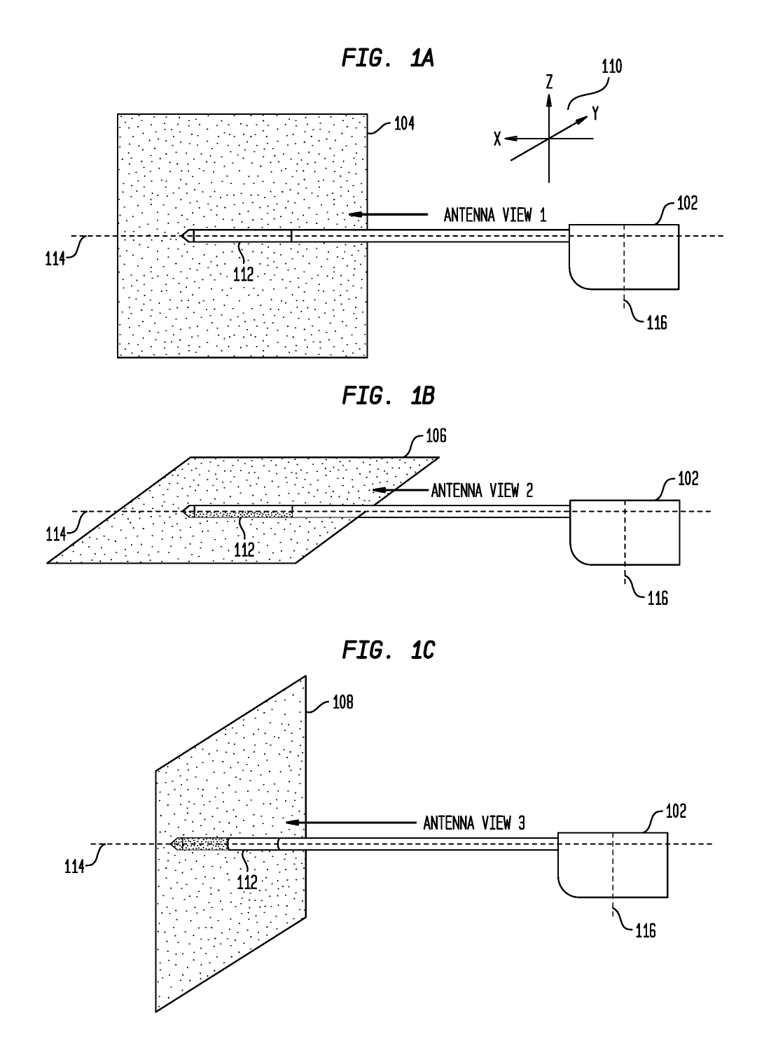

Referring to FIGS. 1A-1C, there is shown an illustration of an ablation probe 102 and various visualization planes 104-108 relative to the ablation probe 102. The visualization planes 104-108 define different views of the anatomy of a patient relative to the position and orientation of an ablation probe 102. In various embodiments, the position and orientation of the ablation probe 102 can be specified as part of a planning operation for an ablation procedure. The planning operation can be based on CT image data, which can be used to visualize the anatomy of a patient. When the CT image data is visualized, a simulated ablation probe 102 can be introduced into the visualization at various positions and orientations.

The CT image data can have a coordinate system 110 for identifying particular locations within the anatomy of the patient. In various embodiments, the coordinate system 110 can be a Cartesian coordinate system having orthogonal X, Y, and Z axes, such as the axes illustrated in FIG. 1A. In various embodiments, the coordinate system can be a spherical coordinate system, which persons skilled in the art will understand as having a radial coordinate and two angular coordinates. In various embodiments, the coordinate system can be another coordinate system. In accordance with one aspect of the disclosed technology, the position of the ablation probe 102 can be specified in the coordinate system 110 using a position coordinate (xₚ, yₚ, zₚ). In various embodiments, the position coordinate can correspond to the center of an ablation probe antenna 112. In various embodiments, the position coordinate can correspond to another reference point in the ablation probe, such as the tip of an ablation antenna. Other reference points in an ablation probe can serve as a position point for visualization and are contemplated to be within the scope of the present disclosure.

As shown in FIG. 1A, the ablation probe 102 defines a central axis 114 and a vertical axis 116. In accordance with an aspect of the present disclosure, the orientation of the ablation probe 102 can be indicated using a vector \vec{v_{c}}=\vec{x_{c}}+\vec{y_{c}}+\vec{z_{c}} that aligns with the central axis 114 of the ablation probe 102 and a vector \vec{vₐ}=\vec{xₐ}+\vec{yₐ}+\vec{zₐ} that aligns with the vertical axis 116 of the ablation probe 102. As mentioned above, the visualization planes 104-108 of FIGS. 1A-1C are oriented relative to the ablation probe 102. As referred to herein, a probe-axial plane 108 (FIG. 1C) refers to the plane that includes the position coordinate (xₚ, yₚ, zₚ) and is orthogonal to the vector \vec{v_{c}} 114, a probe-sagittal plane 104 (FIG. 1A) refers to the plane that includes the position coordinate (xₚ, yₚ, zₚ) and also includes both vectors \vec{v_{c}} 114 and \vec{vₐ} 116, and a probe-coronal plane 106 (FIG. 1B) refers to a plane that includes the position coordinate (xₚ, yₚ, zₚ) and is orthogonal to the vector \vec{vₐ} 116. In one aspect of the present disclosure, the three planes 104-108 are mutually orthogonal. As persons skilled in the art will recognize, the probe-axial 108, probe-sagittal 104, and probe-coronal 106 planes defined herein are different from the axial, sagittal, and coronal planes typically defined for a human body, in that the probe-based planes 104-108 are relative to the orientation of an ablation probe 102 rather than the orientation of the human body. Systems and methods for using these planes to generate views of the anatomy of a patient are described below herein.

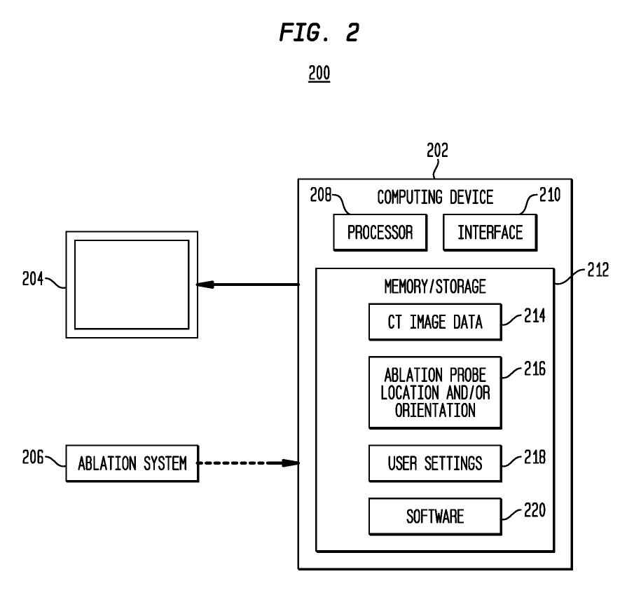

Referring now to FIG. 2, there is shown a block diagram of a system 200, which includes a computing device 202 such as, for example, a laptop, desktop, workstation, tablet, or other similar device, a display 204, and an ablation system 206. The computing device 202 includes one or more processors 208, interface devices 210 (such as communications interface and user interface), memory and storage 212, and/or other components generally present in a computing device. The display 204 may be touch sensitive, which enables the display to serve as both an input and output device. In various embodiments, a keyboard (not shown), mouse (not shown), or other data input devices may be employed.

Memory/storage 212 may be any non-transitory, volatile or non-volatile, removable or non-removable media for storage of information such as computer-readable instructions, data structures, program modules or other data. In various embodiment, the memory 212 may include one or more solid-state storage devices such as flash memory chips or mass storage devices. In various embodiments, the memory/storage 212 can be RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium which can be used to store the desired information and which can be accessed by the computing device 202.

Computing device 202 may also include an interface device 210 connected to a network or the Internet via a wired or wireless connection for the transmission and reception of data. For example, computing device 202 may receive computed tomographic (CT) image data 214 of a patient from a server, for example, a hospital server, Internet server, or other similar servers, for use during surgical ablation planning. Patient CT image data 214 may also be provided to computing device 202 via a removable memory.

In the illustrated embodiment, the memory/storage 212 includes CT image data 214 for one or more patients, information regarding the location and orientation of an ablation probe 216, various user settings 218 (which are described below), and various software that perform the operations described herein 220.

In various embodiments, the system 200 includes an ablation system 206 that includes a generator (not shown) and an ablation probe that includes an ablation antenna, such as the ablation probe 102 shown in FIGS. 1A-1C. The ablation system 206 will be described in more detail later herein.

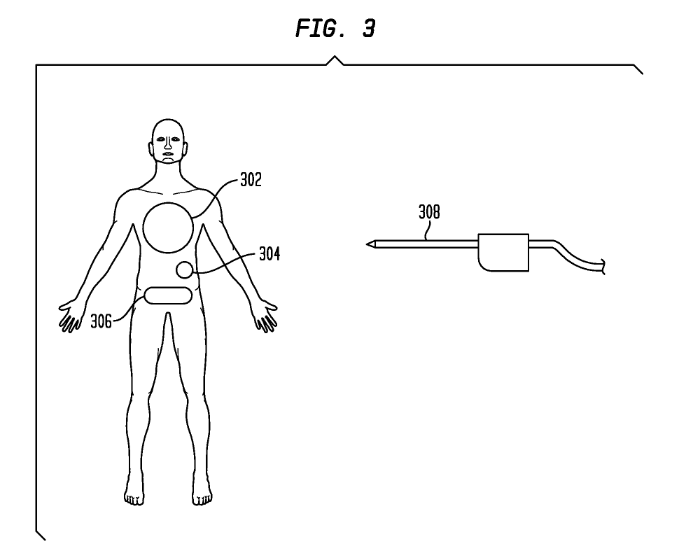

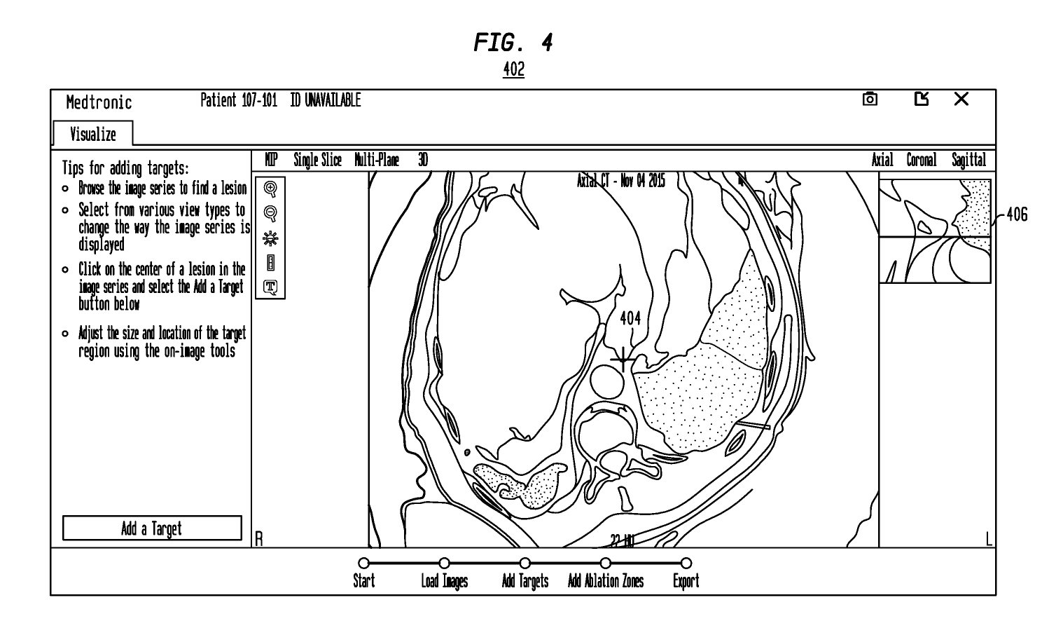

In accordance with an aspect of the present disclosure, the software 220 of FIG. 2 includes a treatment planning module which guides a clinician in identifying a target for ablation treatment, a target size, a treatment zone, and an access route to the target. As used herein, the term “clinician” refers to any medical professional (e.g., doctor, surgeon, nurse, physician assistant, technician, or the like) for planning, performing, monitoring and/or supervising a medical procedure involving the use of the embodiments described herein. The treatment planning module can generate a user interface screen for presenting information and receiving clinician input. The clinician can select a patient data set corresponding to a patient via the user interface. With reference also to FIG. 3, a patient data set can be selected based on, for example, a region where the ablation target is located, such as a lung region 302, a liver region 304, a kidney region 306, or another region of the patient. The patient data set includes CT image data for the selected region 302-306, which will be described below in connection with FIGS. 4-7.

As persons skilled in the art will understand, CT image data are x-ray scans of “slices” of a patient's anatomy. Although each slice views the anatomy from a particular angle, image data across multiple “slices” can be used to generate views of the anatomy from other angles. In particular, as described in connection with FIGS. 1A-1C, based on the position and orientation of an ablation probe 102, an image of the anatomy can be generated for a probe-axial view 108, a probe-sagittal view 104, and a probe-coronal view 106. FIGS. 4-6 illustrate examples of these different views for a lung region 302. The lung region 302 is merely exemplary, and as mentioned above, other regions of the body can be viewed as well.

Referring to FIG. 4, there is shown an image of an axial view 402 of a portion of a lung region generated from CT image data. In various embodiments, the axial view 402 is a probe-axial view. In various embodiments, the axial view 402 is a patient-centric axial view. A clinician can use the software user interface to designate a location 404 in the patient's anatomy at which to generate the axial view 402. The location 404 can be any location encompassed by the CT image data and can be, for example, the location of an ablation target, the center of an ablation zone, or the location of a simulated ablation probe during an ablation planning operation. Additionally, the clinician can use the software user interface to designate an orientation at which to generate a probe-axial view. The orientation can be any direction and can correspond to the orientation of the central axis of a simulated ablation probe during an ablation planning operation. In the illustrated embodiment, a smaller image of the anatomy at another plane can be simultaneously shown, such as a smaller image 406 of a coronal view.

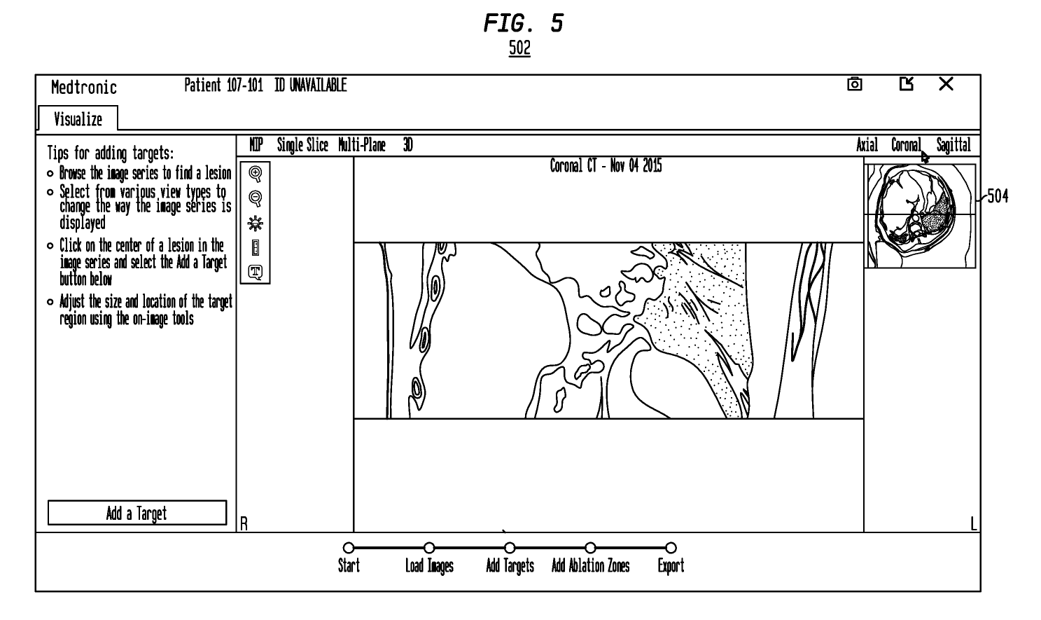

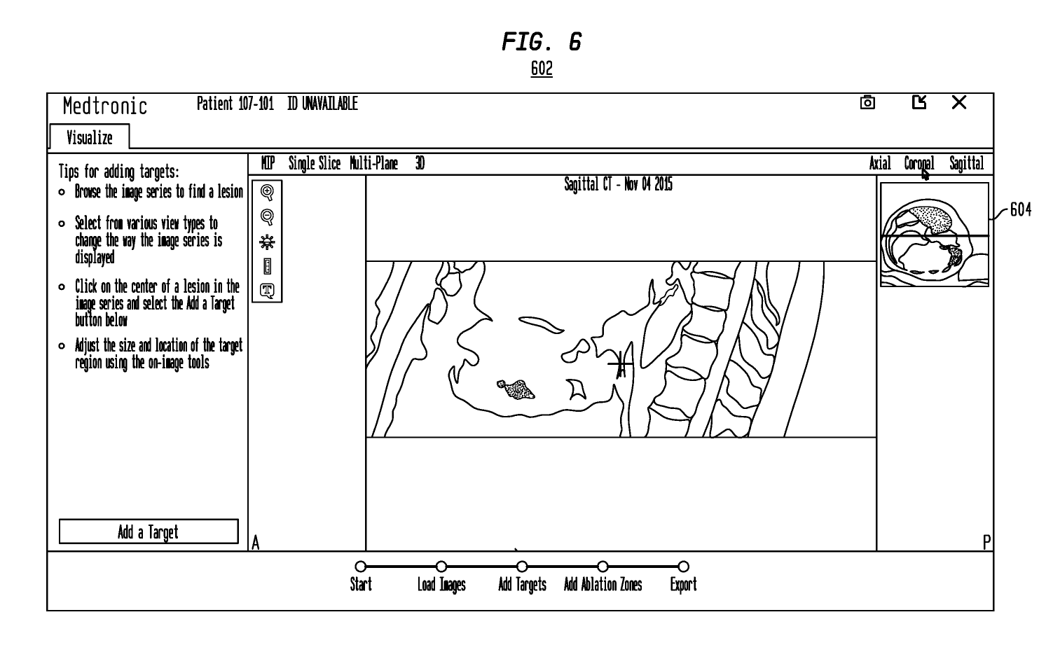

FIG. 5 is an image of a coronal view 502 of a portion of the lung region generated from CT image data, with a smaller image 504 of the axial view shown in the upper right corner. In various embodiments, the coronal view 502 is a probe-coronal view. In various embodiments, the coronal view 502 is a patient-centric coronal view. FIG. 6 is an image of a sagittal view 602 of a portion of the lung region generated from CT image data, with a smaller image 604 of the axial view shown in the upper right corner. In various embodiments, the sagittal view 602 is a probe-sagittal view. In various embodiments, the sagittal view 602 is a patient-centric sagittal view. As with the axial view in FIG. 4, a clinician can use the software user interface to designate a location and an orientation at which to generate the views in FIGS. 5 and 6.

In accordance with one aspect of the present disclosure, in any of the views of FIGS. 4-6, a clinician can interact with the image to move the location and orientation. For example, a clinician can drag the center of the image to move the location at which to center the view, and the clinician can drag the corners or sides of the image to rotate the orientation at which to generate the view. When the clinician designates a new location and/or orientation, the various views can be generated based on the new location and/or orientation.

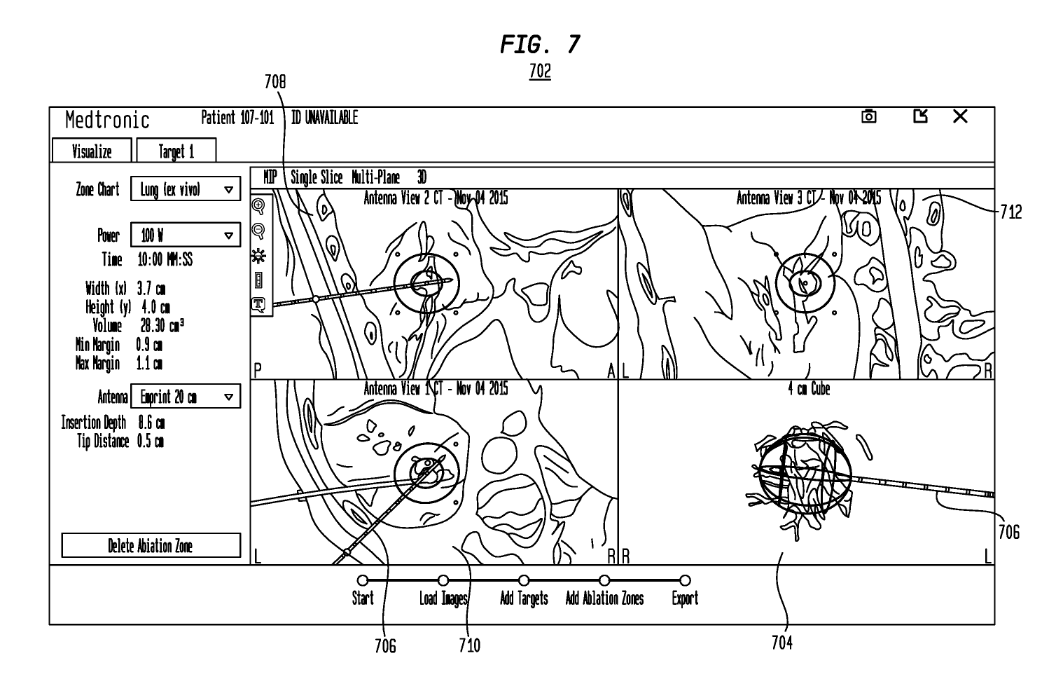

Referring to FIG. 7, there is shown an image 702 of a composite of different views based on a location in a patient's anatomy and an orientation. The illustrated image 702 includes a probe-sagittal view, a probe-coronal view, and a probe-axial view. The illustrated image also includes a three-dimensional depiction 704 of the location within the patient's anatomy. In various embodiments, the three-dimensional depiction 704 can be a depiction of the patient's anatomy within a cubical space. In various embodiments, other geometric spaces can be used for the three-dimensional depiction 704. In various embodiments, the image 702 can include one or more of patient-centric sagittal, coronal, and/or axial views. In various embodiments, the image 702 can include a combination of probe-centric and patient-centric sagittal, coronal, and/or axial views. As shown in FIG. 7, the images also include a depiction of the ablation probe 706 at the location within the patient's anatomy. The ablation probe 706 is depicted based on the selected orientation. In the probe-coronal 708 and probe-sagittal 710 views, the ablation probe 706 is visible because those planes include the central axis of the ablation probe 706. In the probe-axial view 712, the ablation probe 706 is not visible because the probe-axial plane 712 is normal to the central axis of the ablation probe 706.

In various embodiments, the software (220, FIG. 2) can perform an operation to identify similar types of tissue, such as skin, bones, or muscle, or other critical structures such as the lungs, and different types of tissue may be presented differently. Because different tissue types appear with different intensities in a CT image, due to differences in tissue density, different tissue types can be identified by analyzing the intensity values in the CT image. In various embodiments, critical structures of the patient's anatomy may be presented to a physician using a different color, texture, and/or transparency. This presentation provides the clinician with an easier way of viewing different structures and organs within the image. In various embodiments, the different colors, textures, or transparencies can be set by the clinician, and these settings can be stored in the memory/storage of the computing device.

As persons skilled in the art will recognize, techniques for identifying different types of tissue and organs from CT image data include, without limitation, binary masking, determination of the optimum threshold that separates tissue and background, adaptive region growing, wavefront propagation, automatic or manual determination of seed points in critical structures, a fill holes algorithm for filling in holes in the binary mask by flood filling the background and inverting the result, a rolling ball algorithm to close the airways, blood vessels, and indentations corresponding to peripheral nodules, and a morphological closing operation. The operation may also identify tumors automatically and present it different from the surrounding tissue, and may present the clinician.

As mentioned above in connection with FIG. 7, the composite image 702 includes a three-dimensional (3D) depiction 704 or representation of the patient's anatomy. A variety of three-dimensional rendering techniques can be utilized to generate all or part the 3D model. For example, surface rendering is a technique that applies binary masks and various filters to generate a surface mesh. Examples of well-known filters used in surface rendering include dilation filters, masking filters, Gaussian filters, and contour filters. A 3D surface rendered image of a lung or another part of the patient's anatomy can generated by using different combinations of filters and algorithms, such as a marching cubes algorithm or an image smoothing filter, which will be recognized by persons skilled in the art. As shown in FIG. 7, a depiction of an ablation probe 706 can be shown in the 3D representation 704 at the location and orientation indicated by the clinician.

What have been described above are systems and methods for ablation visualization. The following will describe various visualization options during an ablation planning operation conducted using the software 220 of the computing device 202.

In one aspect of the present disclosure, an ablation planning operating may involve adding a target location for ablation treatment. The planning operation may involve the lungs, liver, kidneys, or another anatomical structure. In the add target operation, the probe-centric and/or patient-centric axial, sagittal, and coronal views can be presented without a depiction of the ablation probe, and each view can be displayed individually, as shown in FIGS. 4-6, or simultaneously in a composite image. A 3D depiction can be presented during an add target operation, either by itself or in a composite image as shown in FIG. 7. In various embodiments, the 3D depiction can present skin, muscle, and bone differently. In the case of a planning operation involving the lungs, the lungs can be presented in a different way, as described above herein. Additionally, in the case of the lungs, the 3D depiction can represent the patient's anatomy within a cubical space, such as a four centimeter cubical space. In various embodiments, the software (220, FIG. 2) can present the clinician with the option to select one or more of the probe-axial view, the probe-coronal view, the probe-sagittal view, the three-dimensional depiction, and the cubical space depiction, for display on a display device.

In one aspect of the present disclosure, an ablation planning operating may involve adding an ablation zone for ablation treatment. In the add ablation zone operation, the probe-centric and/or patient-centric axial, sagittal, and coronal views can be presented with or without a depiction of the ablation probe, and each view can be displayed individually or displayed simultaneously in a composite image, as shown in FIG. 7. A 3D depiction can be presented during an add ablation zone operation, either by itself or in a composite image as shown in FIG. 7. In various embodiments, the 3D depiction can present skin, muscle, and bone differently. In the case of a planning operation involving the lungs, the lungs can be presented in a different way, as described above herein. Additionally, in the case of the lungs, the 3D depiction can represent the patient's anatomy within a cubical space, such as a four centimeter cubical space. In various embodiments, the software can present the clinician with the option to select one or more of the axial view, the coronal view, the sagittal view, the three-dimensional depiction, and the cubical space depiction, for display on a display device.

What have been described above are systems and methods for ablation visualization in an ablation planning operation. In one aspect of the present disclosure, ablation visualization is provided in connection with an ablation procedure in real-time. Referring again to FIGS. 2 and 3, and as mentioned above, an ablation system 206 can include a generator (not shown) and an ablation probe 308 connected to the generator. The generator can provide ablation energy to the probe 308, which delivers the energy to target tissue 302-306. In various embodiments, the ablation probe 308 can include sensors that detect the position and orientation of the ablation probe 308. Such capability can be provided by, for example, a six degree-of-freedom inertial measurement unit (IMU) that is commercially available and that can be incorporated into the ablation probe. The position and orientation of the ablation probe 308 in the patient during ablation treatment can be determined in real-time, based on measurements from the IMU, by the ablation system 206 or by the computing device 202. Then, the position and orientation of the ablation probe 308 in the patient during the ablation treatment can be converted to a position and orientation in the CT image data 214 using technology known by persons skilled in the art as antenna-to-CT registration. Based on the position and orientation and the antenna-to-CT registration, a real-time depiction of an ablation probe can be overlaid onto the CT image data to provide a clinician with a real-time visualization of an ablation treatment procedure.

After an ablation treatment has been completed, the clinician may wish to review the difference between a patient's pre-treatment CT image data and post-treatment CT image data. This may be beneficial where repeated treatments are necessary, such as where treatments must be made successively to avoid damaging particular structures such as blood vessels and the like. In one aspect of the present disclosure, a clinician can use the software 220 of the computing device 202 to perform a comparison operation. In the comparison operation, the probe-centric and/or patient-centric axial, sagittal, and coronal views of the pre-treatment CT images and of the post-treatment CT images can be presented without a depiction of the ablation probe, and each view can be displayed individually or displayed simultaneously in a composite image. A 3D depiction can be presented during a comparison operation, either by itself or in a composite image. In various embodiments, the 3D depiction can present skin, muscle, and bone differently.

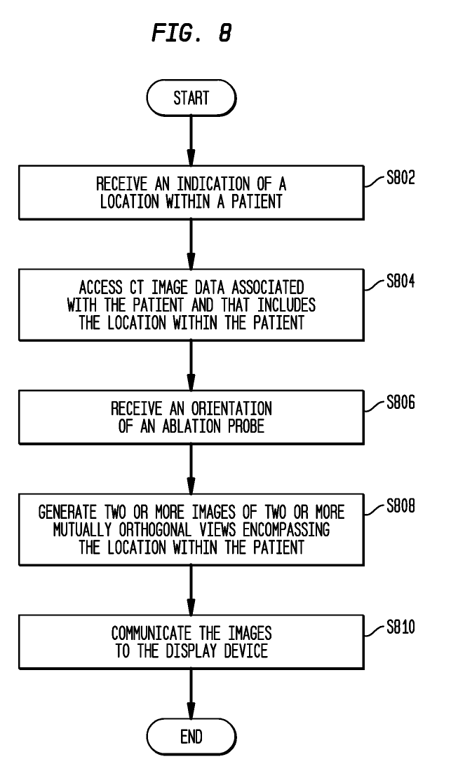

Referring now to FIG. 8, there is shown a flow diagram of an exemplary ablation visualization operation. At S802, the computing device receives an indication of a location within a patient. The location can be received as part of an ablation planning operation or can be a real-time location within a patient received and determined based on location sensing in an ablation probe. At S804, the computing device accesses CT image data that is associated with the patient and that includes image data for the location within the patient. At S806, the computing device receives an orientation of an ablation probe. At S808, the computing device generates, based on the CT image data, two or more images of two or more mutually orthogonal views of the anatomy of the patient. The two or more views encompass the location within the patient, and include at least a probe-axial view of the anatomy of the patient. As described above herein, the probe-axial view is orthogonal to the central axis of the ablation probe. At S810, the computing device communicates the images to a display device for display on the display device.

Although the present disclosure has been described in terms of specific illustrative embodiments, it will be readily apparent to those skilled in the art that various modifications, combinations, rearrangements, and substitutions may be made without departing from the spirit and scope of the present disclosure, as defined by the claims appended hereto.

What is claimed is:

1. A system for ablation visualization, the system comprising:
a display device; and
a computing device communicatively coupled to the display device, the computing device including a processor and a memory storing instructions which, when executed by the processor, cause the computing device to:
receive an indication of a location within a patient;
access CT image data associated with the patient, the CT image data including image data for the location within the patient;
receive an orientation of an ablation probe having a central axis;
generate, based on the CT image data, at least two images of at least two mutually orthogonal views of an anatomy of the patient encompassing the location within the patient, the at least two images including a probe-axial view of the anatomy of the patient, wherein the probe-axial view is orthogonal to the central axis; and
communicate the at least two images to the display device for display on the display device.

2. The system of claim 1, wherein in generating at least two images of at least two mutually orthogonal views of the anatomy of the patient, the instructions, when executed by the processor, further cause the computing device to:
generate, based on the CT image data, an image of a probe-sagittal view of the anatomy of the patient encompassing the location within the patient; and
generate, based on the CT image data, an image of a probe-coronal view of the anatomy of the patient encompassing the location within the patient,
wherein the probe-axial view, the probe-sagittal view, and the probe-coronal view are mutually orthogonal.

3. The system of claim 2, wherein the instructions, when executed by the processor, further cause the computing device to:
generate a composite image including the probe-axial view, the probe-sagittal view, and the probe-coronal view; and
communicate the composite image to the display device for display on the display device.

4. The system of claim 2, wherein:
generating the at least two images including a probe-axial view of the anatomy of the patient includes generating the image of the probe-axial view to include a depiction of the ablation probe with the orientation and positioned at the location within the patient;
generating the image of the probe-sagittal view of the anatomy of the patient includes generating the image of the probe-sagittal view to include a depiction of the ablation probe with the orientation and positioned at the location within the patient, and
generating the image of the probe-coronal view of the anatomy of the patient includes generating the image of the probe-coronal view to include a depiction of the ablation probe with the orientation and positioned at the location within the patient.

5. The system of claim 2, wherein the instructions, when executed by the processor, further cause the computing device to generate, based on the CT image data, a three-dimensional depiction of the anatomy of the patient encompassing the location within the patient.

6. The system of claim 5, wherein generating the three-dimensional depiction of the anatomy of the patient includes generating the three-dimensional depiction to include a depiction of the ablation probe with the orientation and positioned at the location within the patient.

7. The system of claim 5, wherein the instructions, when executed by the processor, further cause the computing device to present to a user a choice to select at least one of the probe-axial view, the probe-sagittal view, the probe-coronal view, or the three-dimensional depiction, for display on the display device.

8. The system of claim 5, wherein generating the three-dimensional depiction of the anatomy of the patient includes generating a depiction of the anatomy of the patient within a cubical space.

9. The system of claim 8, wherein the instructions, when executed by the processor, further cause the computing device to present to a user a choice to select at least one of the probe-axial view, the probe-sagittal view, the probe-coronal view, or the depiction of the anatomy of the patient within the cubical space, for display on the display device.

10. The system of claim 1, wherein the instructions, when executed by the processor, further cause the computing device to receive a planning operation for an ablation procedure based on the CT image data, wherein the indication of the location within the patient and the orientation of the ablation probe are received during the planning operation and are based on the CT image data.

11. The system of claim 1, wherein the planning operation includes at least one of an operation to add an ablation target or an operation to add an ablation zone.

12. The system of claim 1, further comprising the ablation probe, wherein the indication of the location within the patient and the orientation of the ablation probe are based on a real-time location and a real-time orientation of the ablation probe within the patient during a medical procedure.

13. A method for ablation visualization, the method comprising:
receiving an indication of a location within a patient;
accessing CT image data associated with the patient, the CT image data including image data for the location within the patient;
receiving an orientation of an ablation probe having a central axis;
generating, based on the CT image data, at least two images of at least two mutually orthogonal views of an anatomy of the patient encompassing the location within the patient, the at least two images including a probe-axial view of the anatomy of the patient, wherein the probe-axial view is orthogonal to the central axis; and
communicating the at least two images to a display device for display on the display device.

14. The method of claim 13, wherein generating the at least two images includes:
generating, based on the CT image data, an image of a probe-sagittal view of the anatomy of the patient encompassing the location within the patient; and
generating, based on the CT image data, an image of a probe-coronal view of the anatomy of the patient encompassing the location within the patient,
wherein the probe-axial view, the probe-sagittal view, and the probe-coronal view are mutually orthogonal.

15. The method of claim 14, further comprising:
generating a composite image including the probe-axial view, the probe-sagittal view, and the probe-coronal view; and
communicating the composite image to the display device for display on the display device.

16. The method of claim 14, further comprising generating, based on the CT image data, a three-dimensional depiction of the anatomy of the patient encompassing the location within the patient.

17. The method of claim 14, further comprising presenting to a user a choice to select at least one of the probe-axial view, the probe-sagittal view, the probe-coronal view, or the three-dimensional depiction, for display on the display device.

18. The method of claim 13, further comprising receiving a planning operation for an ablation procedure based on the CT image data, wherein the indication of the location within the patient and the orientation of the ablation probe are received during the planning operation and are based on the CT image data.

19. The method of claim 18, wherein the planning operation includes at least one of an operation to add an ablation target or an operation to add an ablation zone.

20. The method of claim 13, wherein the indication of the location within the patient and the orientation of the ablation probe are based on a real-time location and a real-time orientation of the ablation probe within the patient during a medical procedure.
